(19) United States
(12) Patent Application Publication
(10) Pub. No.: US 2006/0247482 A1
(43) Pub. Date: Nov. 2, 2006

(54) Method and system to recycle non-isomerized monomer in an ionic liquid catalyzed chemical reaction

(75) Inventors: Kenneth D. Hope, Kingwood, TX (US); Donald A. Stern, Kingwood, TX (US)

Correspondence Address: CHEVRON PHILLIPS CHEMICAL COMPANY LP, LAW DEPARTMENT - IP, P.O BOX 4910, THE WOODLANDS, TX, 77387-4910, US

(73) Assignee: Chevron Phillips Chemical Company

(21) Appl. No.: 11/264,412

(22) Filed: Nov. 1, 2005

Related U.S. Application Data
(60) Provisional application No. 60/676,545, filed on Apr. 29, 2005.

(51) Int. Cl. C07C 2/02

(52) U.S. Cl. 585/521

(57) ABSTRACT

In an embodiment, a method to produce an oligomer using a recycle stream that contains a substantially non-isomerized monomer. The monomer is contacted with an ionic liquid catalyst to produce the oligomer. The monomer includes at least one alpha olefin. Along with the substantially non-isomerized monomer, a dimer can also be recycled. At least a portion of the catalyst can be recycled also.

### CROSS-REFERENCE TO RELATED APPLICATIONS

The present application claims the benefit under the provisions of 35 U.S.C. §119(e) of U.S. Provisional Application No. 60/676,545 filed Apr. 29, 2005 and entitled “Method and System-to Recycle Non-Isomerized Monomer in an Ionic Liquid Catalyzed Chemical Reaction,” which hereby is incorporated by reference in its entirety.

### FIELD OF THE INVENTION

The present invention generally relates to ionic liquid catalytic systems for chemical conversions. More specifically, the invention relates to increased activity of ionic liquid catalysts for increased monomer conversion in the manufacture of polyalphaolefin products.

### BACKGROUND

Ionic liquid catalysts can be used to catalyze a variety of chemical reactions, for example the oligomerization of alpha olefins to produce polyalphaolefins (PAO). A polyalphaolefin is a synthetic hydrocarbon liquid that is typically manufactured from the oligomerization of C₆ to C₂₀ alpha olefins. Polyalphaolefins are used in various industries as lubricants in gear oils, greases, engine oils, fiber optic gels, transmission oils, and various other lubricant applications. Ionic liquid catalysts used to produce PAO can be quite costly. Therefore, there is a need in the art for a method to increase the activity of an ionic liquid catalyst, for example to reduce the amount of required catalyst and still maintain the desired conversion, thereby improving economics of a process.

### SUMMARY OF THE INVENTION

In an embodiment, a method is disclosed to produce an oligomer comprising the steps of contacting a monomer comprising an alpha olefin with an ionic liquid catalyst to produce a reactor effluent stream The effluent stream comprises at least a portion of the monomer, the oligomer, the ionic liquid catalyst, and combinations thereof. The effluent stream is then separated to isolate or remove at least a portion of the monomer from the oligomer and the ionic liquid catalyst. At least a portion of the monomer is recycled for use in the step of contacting the monomer with the ionic liquid catalyst. The at least a portion of the monomer is substantially non-isomerized through the step of contacting the monomer with the ionic liquid catalyst.

In an aspect, the recycle monomer stream can also include a dimer. One or more separation steps can be used to separate the unreacted monomer and the dimer from the reactor effluent stream. In another aspect, at least a portion of the liquid ionic catalyst can be recycled, along with at least a portion of the monomer, for use in the step of contacting the monomer with the ionic liquid catalyst.

In some embodiments, the alpha olefin comprises from 4 to 20 carbons; alternatively, from 6 to 20 carbon atoms; alternatively, from 8 to 16 carbon atoms; and alternatively, from 10 to 14 carbon atoms. In some embodiments, the alpha olefin is 1-decene, 1-dodecene, or combinations thereof. In some embodiments, the alpha olefin is 1-decene.

In an aspect, the ionic liquid catalyst comprises a metal halide and an alkyl-containing amine hydrohalide salt. In another aspect, the step of contacting the monomer with the ionic liquid catalyst includes contacting the monomer with the ionic liquid catalyst and oxygen, water, or combinations thereof.

In an aspect, the methods described herein can also include the step of hydrogenating the oligomer to produce a polyalphaolefin product.

### BRIEF SUMMARY OF THE DRAWINGS

FIG. 1 is a process flow schematic of one embodiment of the system to add high shear mixing to an ionic liquid catalyzed reaction incorporated within a process for manufacturing a polyalphaolefin product;

FIG. 2 contains ¹³C NMR data from a monomer recycle stream from a prior art process used to produce polyalphaolefin; and

FIG. 3 contains ¹³C NMR data from a monomer recycle stream from a process according to an embodiment of the present invention.

### DETAILED DESCRIPTION

The invention relates to a method and a system to produce an oligomer using an ionic liquid catalyst in combination with a monomer recycle stream that comprises unreacted monomer from the feed monomer that remains substantially non-isomerized during contacting a monomer feedstock with the liquid ionic catalyst. More specifically, the invention relates to a process to produce the oligomer comprising the step of contacting the monomer feedstock with the ionic liquid catalyst to produce a reactor effluent stream. The reactor effluent stream comprises at least a portion of the monomer feedstock, the oligomer, the ionic liquid catalyst, and combinations thereof The effluent stream is then separated in one or more separation steps to isolate or remove at least a portion of the monomer from the oligomer and the ionic liquid catalyst. At least a portion of the monomer is recycled for use in the step of contacting the monomer with the ionic liquid catalyst. The at least a portion of the monomer is substantially non-isomerized through the step of contacting the monomer with the ionic liquid catalyst. The monomer feed, ionic liquid catalyst, methods of contacting the monomer feedstock with the ionic liquid catalyst, and other process parameters are described herein. In an aspect, the oligomer is hydrogenated to produce a polyalphaolefin product.

In some embodiments of such a polyalphaolefin process, the ionic liquid catalyst is contacted with oxygen. In other embodiments, the ionic liquid catalyst is contacted with water. In yet other embodiments, the ionic liquid catalyst is contacted with oxygen and water. The monomer feedstock, ionic liquid catalyst, quantity of oxygen and/or water, and other process parameters are described herein.

The following disclosure primarily focuses on the implementation of the invention to the production of PAOs. However, it should be understood that the scope of the present invention is defined by the claims and not limited to a particular embodiment described herein. Thus, the invention described herein can be equally applied to an alkylation reaction, an olefin polymerization reaction, or an olefin oligomerization reaction, for example.

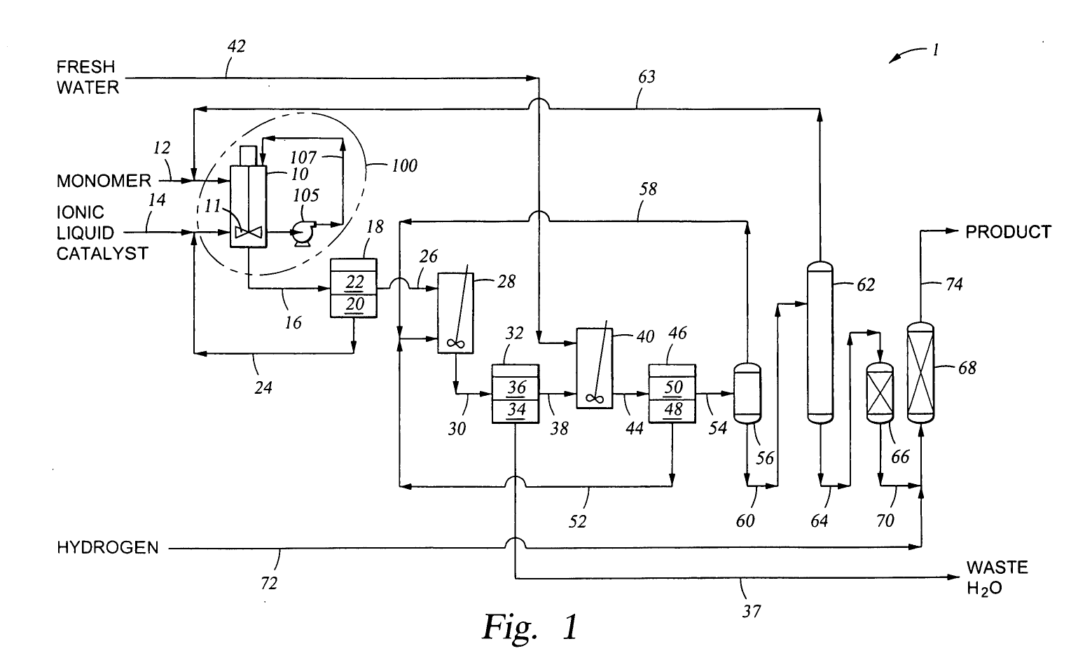

FIG. 1 depicts a system 100 to use the recycle monomer stream in an ionic liquid catalyzed process 1 for manufacturing a hydrogenated polyalphaolefin (PAO) product. The system 3100 comprises a reactor 10. Liquid reactant feed stream 12 and ionic liquid catalyst stream 14 are fed into a reaction zone within the reactor 10. The reactor 10 can be any means known in the art for contacting the reactants with the ionic liquid under conditions described herein. Examples of suitable reactors include stirred tank reactors, which can be either a batch reactor or continuous stirred tank reactor (CSTR). Alternatively, tubular or loop reactors can be employed and equipped with suitable means for emulsifying as described herein. A reaction effluent comprising one or more reaction products can be withdrawn from reactor 10 via product line 16.

The reaction that occurs within the reaction zone can be an oligomerization reaction. In an embodiment, the reaction zone of system 100 comprises an oligomerization reaction in reactor 10 wherein reactant feed stream 12 comprises alpha-olefin monomer and product line 16 comprises a polyalphaolefin (PAO) product. Non-limiting examples of suitable alpha olefin monomers include alpha olefins having 4 to 20 carbon atoms; alternatively, 6 to 20 carbon atoms; alternatively, 8 to 16 carbon atoms; and alternatively, 10 to 14 carbon atoms.

The reactants and ionic liquid catalyst can be introduced separately into the reaction zone via separate feed streams, as shown in FIG. 1, or they can be introduced together as a premixed mixture Liquid components within the reaction zone include the liquid reactants such as monomer, reaction products (e.g., PAO, dimer, etc.), and optionally one or more solvents. The reactants and the ionic liquid catalyst are generally immiscible fluids, such that if simply poured together, they would form two layers of material with the more dense of the two (typically the catalyst) settling on the bottom. The amount of contact between the reactants and catalyst would be severely limited in this scenario to merely the interface between the two layers. Therefore the reactor 10 can be equipped with one or more means for emulsifying the liquid components and ionic liquid catalyst. Generally, emulsifying reduces an ionic liquid catalyst droplet size, thereby increasing a surface area of the ionic liquid catalyst available for contact in the reaction zone. The methods and systems for emulsifying the liquid components and ionic liquid catalyst are described in U.S. patent application Ser. No. 10/978,792 filed Nov. 1, 2004 and entitled “Method and System to Add High Shear to Improve an Ionic Liquid Catalyzed Chemical Reaction,” the disclosure of which is incorporated herein by reference in its entirety.

The reaction conditions within the reaction zone are maintained so as to provide suitable reaction conditions for the oligomerization of the alphaolefin of the monomer feed to give a desired polyalphaolefin product. The reaction pressure generally can be maintained in the range of from: below atmospheric upwardly to about 250 psig. Because the reaction is not significantly pressure dependent, it is most economical to operate the reactor at a low pressure, for example, from about atmospheric to about 50 psig and, alternatively, from atmospheric to 25 psig. The reaction temperature is to be maintained during the reaction so as to keep the reactants and catalyst: in the liquid phase. Thus, generally, the reaction temperature range is from about 20° F. to about 200° F. In an embodiment, the reaction temperature is in the range of from about 40° F. to about 150° F., and, alternatively, from 50° F. to 110° F.

The residence time of the feed within the reaction zone has a small influence on the resultant reaction product. As used herein, the term “residence time” is defined as being the ratio of the reactor volume to the volumetric introduction rate of the feeds, both the monomer feed and the ionic liquid catalyst feed, charged to or introduced into the reaction zone defined by a reactor. The residence time is in units of time. The reactor volume and feed introduction rate are such that the residence time of the total of the monomer feed and ionic liquid catalyst feed is generally in the range upwardly to about 300 minutes, but due to the need to have sufficient residence time for the reaction to take place and to economic considerations, the residence time is more appropriately in the range of from about 1 minute to about 200 minutes. In an embodiment, the residence time is in the range of from about 2 minutes to about 120 minutes and, alternatively, from 5 minutes to 60 minutes.

The amount of oxygen, the amount of water, or both present in the reaction zone can be controlled as described in previously referenced U.S. patent application Ser. No. 10/978,547 and entitled “Method and System to Contact an Ionic Liquid Catalyst with Oxygen to Improve a Chemical Reaction,” which claims the benefit of and priority to U.S. Provisional Patent Application No. 60/516,516, filed Oct. 31, 2003 and entitled “Method and System to Contact an Ionic Liquid Catalyst with Oxygen to Improve a Chemical Reaction” and U.S. patent application Ser. No. 10/420,261, filed Apr. 22, 2003, and entitled “Method for Manufacturing High Viscosity Polyalphaolefins Using Ionic Liquid Catalysts”.

The catalyst concentration in the reaction zone can be used to control certain desired physical properties of the polyalphaolefin product. In an embodiment, the weight percent of ionic liquid catalyst introduced into the reaction zone can be from about 0.1 to about 50 wt. % based on the weight of the feed to the reactor; alternatively, from about 0.1 to about 25 wt. %; alternatively, from about 0.1 to about 10 wt. %; alternatively, from about 0.1 to about 5 wt. %; alternatively, from about 1 to about 3 wt. %; alternatively, from about 1.5 to about 2.5 wt. %; and alternatively, from about 2.0 to about 2.5 wt. %. In an embodiment, the weight percent of ionic liquid catalyst introduced into the reaction zone is less than about 7.5 wt. % based upon the weight of the feed to the reactor. In an alternate embodiment, shear pump 105 can be operated at a high shear rate of from about 20,000 to about 60,000 sec⁻¹. In this embodiment, the weight percent of the ionic liquid catalyst introduced into the reaction zone can be reduced by about 20 percent, for example reduced from about 2.5 wt. % of catalyst present in the reaction zone to about 2.0 wt. %, to get an equivalent viscosity product.

In the manufacture of polyalphaolefins, the monomer feedstock that is introduced into the reaction zone of the process comprises at least one alpha olefin. In an embodiment, the monomer feed comprises, based on the weight of the monomer feed, at least about 50 weight percent alpha olefins, alternatively, at least about 60, 70, 80, 90, 95, or 99 weight percent alpha olefins. In an embodiment, the monomer feed consists essentially of alpha olefins, which should be understood to include commercially available alpha olefin products. The alpha olefins and combinations thereof, which are also known as 1-olefins or 1-alkenes, suitable for use as the monomer feed of the process can have from 4 to 20 carbon atoms and include, for example, 1-butene, 1-pentene, 1-hexene, 1-octene, 1-decene, 1-dodecene, 1-tetradecene and combinations thereof. In some embodiments, the monomer feed comprises 1-decene. In other embodiments the monomer feed comprises 1-dodecene. In other embodiments, the monomer feed consists essentially of 1-decene, 1-dodecene, or combinations thereof. In an embodiment, the alpha olefins of the monomer feed have from 4 to 20 carbon atoms or mixtures thereof; alternatively, from 6 to 18 carbon atoms; and alternatively, from about 10 to about 12 carbon atoms.

The reactor effluent withdrawn from the reaction zone generally comprises polyalphaolefins and the ionic liquid catalyst. The reactor effluent stream can include unreacted monomer and a dimer of the alpha olefin, as described herein. A variety of polyalphaolefins can be produced according to the present disclosure. Polyalphaolefins are synthetic hydrocarbon liquids manufactured from monomers. Polyalphaolefins have a complex branched structure with an olefin bond, i.e., carbon-carbon double bond that can be located anywhere along the molecule due to isomerization by the catalyst. As used herein, the term “polyalphaolefins” includes an alpha olefin oligomerization product that is either a dimer, a trimer, a tetramer, higher oligomers, a polymer of an alpha olefin, or a mixture of any one or more thereof, each of which has certain desired physical properties and, in particular, having the desired high viscosity properties all of which are more fully described below. Thus, the polyalphaolefins can include dimers, trimers, tetramers, higher oligomers, polymers, or mixture of any one or more thereof of the alpha olefin contained in the monomer feed. Such dimers, trimers, tetramers, higher oligomers, polymers, or mixture of any one or more thereof can comprise molecules having from 12 to over 1300 carbon atoms.

The reactor effluent can further comprise a dimer of the alpha olefin in the monomer feed and the unreacted monomer. In an aspect, the unreacted monomer is substantially non-isomerized, i.e., is essentially free of isomers of the unreacted monomer. The polyalphaolefins can be separated from the other components of the reactor effluent including the ionic liquid catalyst, and, optionally, the unreacted monomer and dimers formed during the reaction of the monomer feed. The separated polyalphaolefins can undergo subsequent processing or upgrading such as hydrogenation to form a more stable polyalphaolefin product (referred to herein as a hydrogenated polyalphaolefin product), for example useful as a base oil stock. Hydrogenated polyalphaolefin products have olefin-carbons saturated with hydrogen, which lends excellent thermal stability to the molecule.

In an embodiment, the hydrogenated polyalphaolefin product has a viscosity of from about 2 to about 100 cSt @ 100° C., e.g., a low viscosity hydrogenated polyalphaolefin product having a viscosity of from about 2 to about 12 cSt @ 100° C., a medium viscosity hydrogenated polyalphaolefin product having a viscosity of from about 12 to about 40 cSt @ 100° C., or a high viscosity hydrogenated polyalphaolefin product having a viscosity of from about 40 to about 100 cSt @ 100° C. The weight average molecular weight of a hydrogenated polyalphaolefin product can be in the range of from about 170 to about 18,200; alternatively, from about 200 to about 10,000; alternatively, from about 210 to about 8,000; and alternatively, from about 250 to about 3,000. In other embodiments, the weight average molecular weight of a hydrogenated polyalphaolefin product can be in the range of from about 500 to about 8,000; alternatively, from about 1,000 to about 5,000; and alternatively, from about 1,500 to 2,500.

In an embodiment, a hydrogenated polyalphaolefin product can be manufactured from either a 1-decene or 1-dodecene feedstock or mixtures thereof The hydrogenated polyalphaolefin products from these feedstocks are especially significant in that they have unique physical properties. Typical ranges for the various physical properties of a hydrogenated polyalphaolefin product and the relevant test methods for determining the physical properties are presented in the following Table 1.

TABLE 1
Hydrogenated PAO Product Physical Properties

| Test | Units | Test Method | Value | |
|---|---|---|---|---|
| Kinematic Viscosity at | cSt | ASTM D445 | Min | 12.0 |
| 100° C. | | | Max | 35.0 |
| Bromine Index | mg/100 g | ASTM D2710 | Max | 800 |
| Volatility, Noack | wt % | CEC L40 T87 | Max | 2.0 |
| Flash Point | ° C. | ASTM D92 | Min | 245 |
| Fire Point | ° C. | ASTM D92 | Min | 290 |
| Pour Point | ° C. | ASTM D97 | Max | −30 |
| Polydispersity Index | | | Max | 3.5 |
| | | | Min | 1.0 |
| Weight Average Molecular | | | Min | 170 |
| Weight | | | Max | 18200 |

Any ionic liquid catalyst suitable to catalyze a desired chemical reaction can be used. Examples of ionic liquid compositions suitable for use in the inventive process are complexes of two components that form compositions that are liquid under the reaction conditions of the inventive process. Specifically, the ionic liquid catalyst is the complex resulting from the combination of a metal halide and an alkyl-containing amine hydrohalide salt. Such compositions are described in detail in U.S. Pat. Nos. 5,731,101 and 6,395,948, the disclosure of each of which is incorporated herein by reference in its entirety. It has been found that the use of such ionic liquid compositions provide for a polyalphaolefin end-products having certain desirable and novel physical properties that make them especially useful in various lubricant or lubricant additive applications. The use of ionic liquid composition to produce polyalphaolefin end-product are described in U.S. Pat. No. 6,395,948 and U.S. patent application Ser. No. 10/900221, filed Jun. 20, 2004, the disclosure of each of which is incorporated herein by reference in its entirety.

The metal halides that can be used to form the ionic liquid catalyst used in this invention are those compounds that can form ionic liquid complexes that are in liquid form at the reaction temperatures noted above when combined with an alkyl-containing amine hydrohalide salt. Examples of suitable metal halides are covalently bonded metal halides. Possible suitable metals which can be selected for use herein include those from Groups IVB, VIII, IB, IIB, and IIIA of the Periodic Table of the Elements, CAS version. More specifically, the metal of the metal halides can be selected from the group consisting of aluminum, gallium, iron, copper, zinc, titanium, and indium; and alternatively, the group consisting of aluminum and gallium, and alternatively, aluminum. Examples of metal halides include those selected from the group consisting of aluminum halide, alkyl aluminum halide, gallium halide, and alkyl gallium halide, titanium halide, alkyl titanium halide, and mixtures thereof of In some embodiments, the metal halide is aluminum halide or alkyl aluminum halide. In an embodiment, the metal halide is aluminum trichloride.

The alkyl-containing amine hydrohalide salts that can be used to form the ionic liquid catalyst used in this invention include monoamines, diamines, triamines and cyclic amines, all of which include one or more alkyl group and a hydrohalide anion. The term alkyl is intended to cover straight and branched alkyl groups having from 1 to 9 carbon atoms. Examples of alkyl-containing amine hydrohalide salts useful in this invention have at least one alkyl substituent and can contain as many as three alkyl substituents. They are distinguishable from quaternary ammonium salts that have all four of their substituent positions occupied by hydrocarbyl groups. Examples include compounds having the generic formula R₃N.HX, where at least one of the “R” groups is an alkyl, for example an alkyl of from one to eight carbon atoms (for example, lower alkyl of from one to four carbon atoms) and X is a halogen, for example chloride. If each of the three R groups is designated R₁, R₂ and R₃, respectively, the following possibilities exist in certain embodiments: each of R₁-R₃ can be lower alkyl optionally interrupted with nitrogen or oxygen or substituted with aryl; R₁ and R₂ can form a ring with R₃ being as previously described for R₁; R₂ and R₃ can either be hydrogen with R₁ being as previously described; or R₁, R₂ and R₃ can form a bicyclic ring. In an embodiment, these groups are methyl or ethyl groups. In certain embodiments, the di- and tri-alkyl species can be used. In other embodiments, one or two of the R groups can be aryl. The alkyl groups and aryl, if present, can be substituted with other groups, such as a halogen. Phenyl and benzyl are representative examples of possible aryl groups to select. However, such further substitution can undesirably increase the viscosity of the melt. Therefore, in an embodiment, the alkyl groups and aryl, if present, can be comprised of carbon and hydrogen groups, exclusively. Such short chains are desired because they form the least viscous or the most conductive melts. Mixtures of these alkyl-containing amine hydrohalide salts can be used.

In an embodiment, the alkyl containing amine hydrohalide salt are those compounds where the R groups are either hydrogen or an alkyl group having 1 to 4 carbon atoms, and the hydrohalide is hydrogen chloride, an example of which is trimethylamine hydrochloride.

The prepared ionic liquid can be stored and subsequently used as a catalyst for the reactions described herein. Once used as a catalyst, the ionic liquid can be separated and/or recovered from the reaction effluent by methods known to those skilled in the art. The separated and/or recovered ionic liquid can be recycled for use as a catalyst either alone or in combination with freshly prepared ionic liquid catalyst. In some cases, the recycled ionic liquid composition can be refortified with a quantity of metal halide or amine hydrohalide salt.

As shown in FIG. 1, the monomer feed and the recycled monomer and dimer, which are more fully described below, are introduced or charged to reactor 10, hereinafter referred to as continuous stirred tank reaction or CSTR 10, by way of feed line 12. Makeup ionic liquid catalyst: and recycled ionic liquid catalyst feed, which is more fully described below, are introduced or charged to CSTR 10 by way of catalyst feed line 14. The monomer and ionic liquid catalyst feeds are introduced into the CSTR 10, blended with stirrer 11, and circulated in circulation loop 107 around CSTR 10. Pump 105, placed within line 107, emulsifies the two immiscible fluids as the fluids are pumped through and returns the emulsion to the CSTR 10 via line 107. The reactor effluent from CSTR 10 is simultaneously withdrawn from CSTR 10 through line 16 as the feeds are being introduced to CSTR 1O.

The reactor effluent is passed from CSTR 10 through line 16 to first phase separator 18 that provides means for separating the reactor effluent into an ionic liquid catalyst phase 20 and a hydrocarbon or polyalphaolefin-containing phase 22. The separated ionic liquid catalyst phase 20 is recycled by way of line 24 and combined with the makeup ionic liquid catalyst passing through line 14 and thereby is introduced into CSTR 10. The first phase separator can be any phase separator able to separate two immiscible liquid having different densities known to those skilled in the art. For example, the first phase separator can be a gravity separator or a centrifugal separator. Other suitable separation means will be apparent to those of skill in the art and are to be considered within the scope of the present invention.

The polyalphaolefin-containing phase 22 passes from phase separator 18 through line 26 to deactivation vessel 28, which provides means for contacting any remaining ionic liquid catalyst mixed with the polyalphaolefin-containing phase with water to deactivate the ionic liquid catalyst. The mixture of polyalphaolefin-containing phase, water, and deactivated ionic liquid catalyst passes from deactivation vessel 28 through line 30 to second phase separator 32. Separator 32 provides means for separating the waste water and catalyst phases 34 and polyalphaolefin containing phase 36. As in all of the separation steps in the present invention, separating can occur in one or more separation steps. The waste water phase passes from second phase separator 32 by way of line 37.

The polyalphaolefin-containing phase 36 passes from second phase separator 32 through line 38 to water wash vessel 40 that provides means for contacting the polyalphaolefin-containing phase 36 with fresh water. The fresh water is charged to or introduced into water wash vessel 40 through line 42. The water and polyalphaolefin-containing phases pass from water wash vessel 40 through line 44 to third phase separator 46. Third phase separator 46 provides means for separating the water and the polyalphaolefin-containing phase introduced therein from water wash vessel 40 into a water phase 48 and polyalphaolefin-containing phase 50. The water phase 48 can be recycled and introduced into deactivation vessel 28 through line 52 thereby providing the deactivation wash water for use in the deactivation vessel 28.

The polyalphaolefin-containing phase 50 passes from third phase separator 46 through line 54 to water separation vessel 56. Water separation vessel 56 provides means for separating water from the polyalphaolefin-containing phase 50, for example by flash separation, to provide a flash water stream and a polyalphaolefin-containing phase having a low water concentration. The flash water stream can pass from water separation vessel 56 and be recycled to deactivation vessel 28 through line 58, or alternatively, the flash water stream can be disposed of as waste water via line 37. The polyalphaolefin-containing phase having a low water concentration passes from water separation vessel 56 through line 60 and is charged to separation vessel 62, which can be, for example, an evaporator. Separation vessel 62 provides means for separating the polyalphaolefin-containing phase having a low water concentration into a first stream comprising monomer and, optionally, dimer, and a second stream comprising a polyalphaolefin product. The first stream passes from separation vessel 62 by way of line 63 and is recycled to line 12 wherein it is mixed with the monomer feed and charged to CSTR 10.

The monomer contained within line 63 that is recycled is substantially non-isomerized as a result of the monomer being reacted in the presence of the ionic liquid catalyst. As used herein, the term “substantially non-isomerized” means that the recycle line 63 contains less than about 15 wt. % isomers of the monomer; alternatively, 10 wt. % isomers of the monomer; alternatively, 7.5 wt. % isomers of the monomer; alternatively, 5 wt. % isomers of the monomer, alternatively, less than about 3 wt. %; alternatively, less than about 2 wt. %; or alternatively, less than 1 wt. %. In prior art polymerization processes, significant amounts of isomers can form from the unreacted feed monomer. When the isomers of the monomers are recycled, the conversion rates for the process suffer. In the present invention, the carbon-carbon double bonds within the feed monomer do not migrate, thereby substantially minimizing or essentially eliminating the formation of isomers during the reaction.

The recycle monomer in line 63 can be recycled to comprise up to 100% of monomer feed line 12. In some embodiments, the recycle monomer feed comprises greater than 10 percent of the total monomer feed; alternatively, greater than 20 weight percent of the total monomer feed; alternatively, greater than 30 weight percent of the total monomer feed; alternatively, greater than 40 weight percent of the total monomer feed; alternatively, greater than 50 weight percent of the total monomer feed; alternatively, greater than 60 weight percent of the total monomer feed; alternatively, greater than 70 weight percent of the total monomer feed; alternatively, greater than 80 weight percent of the total monomer feed; or alternatively, greater than 90 weight percent of the total monomer feed. It is believed that the substantially non-isomerized recycle monomer performs essentially the same as fresh feed monomer in line 12. Viscosity of the final product and process conversion rates are essentially the same as when compared with using fresh monomer, without any recycle monomer. Alternatively, the recycle monomer can be fed at a fresh monomer to recycle monomer feed ratio of about 10:90 to about 90:10; alternatively, from about 25:75 to about 75:25; alternatively, from about 30:70 to about 70:30; alternatively from about 40:60 to about 60:40; or alternatively, from about 50:50.

The second stream passes from separation vessel 62 through line 64 to guard vessel 66. Guard vessel 66 defines a zone containing guard bed material and provides means for removing chlorine and other possible contaminants from the second stream prior to charging it to hydrogenation reactor 68. The effluent from guard vessel 66 passes through line 70 to hydrogenation reactor 68. Hydrogenation reactor 68 provides means for reacting the polyalphaolefin product in the second stream to provide a hydrogenated polyalphaolefin product of which a substantial portion of the carbon-carbon double bonds are saturated with hydrogen. Hydrogen is introduced by way of line 72 into line 70 and mixed with the second stream prior to charging the thus-mixed hydrogen and second stream into hydrogenation reactor 68. The hydrogenated polyalphaolefin product passes from hydrogenation reactor 68 by way of line 74.

The present disclosure primarily focuses on a PAO production embodiment, but it should be understood that the scope of the present invention is defined by the claims and not limited to a particular embodiment described herein. For example, in an alternate embodiment, the reaction zone of system 100 comprises an alkylation reaction in reactor 10 wherein reactant feed stream 12 comprises an aromatic compound such as benzene, toluene, xylene, or naphthalene and product stream 16 comprises an alkylated product.

In an embodiment, the alkylation reaction can be a Friedel-Crafts alkylation. In an embodiment, the alkylation reaction is alkylation of benzene, for example according to the method and apparatus in the U.S. Pat. No. 5,824,832, entitled “Linear Alkylbenzene Formation Using Low Temperature Ionic Liquid”, filed on Oct. 20, 1998, incorporated by reference herein in its entirety. In an embodiment, benzene is alkylated to form ethylbenzene, cumene, or linear alkylbenzenes (LAB). For example, benzene can be combined, typically in molar excess, with a suitable alkylating reagent having from about 2 to 54 carbon atoms such as olefins, halogenated alkanes, or mixtures thereof. Non-limiting examples of suitable halogenated alkanes include C₄-C₂₀ chloroparaffins, alternatively C₁₀-C₁₄ chloroparaffins. Non-limiting examples of suitable olefins include linear, unbranched monoolefins and mixtures thereof having 4 to 20 carbon atoms, alternatively 20 to 24 carbon atoms, alternatively 8 to 16 carbon atoms, and alternatively 10 to 14 carbon atoms, wherein the double bond can be positioned anywhere along the linear carbon chain. Non-limiting examples of other suitable alkylating agents include olefin oligomers such as propylene tetramer and unhydrogenated polyalphaolefins. Ionic liquid catalysts such as those described in more detail herein can be used to catalyze such alkylation reactions.

The following examples of the invention are presented merely for the purpose of illustration and are not intended to limit in any manner the scope of the invention.

### EXAMPLE 1

### Monomer Recycle Stream in Oligomerization of 1-Decene

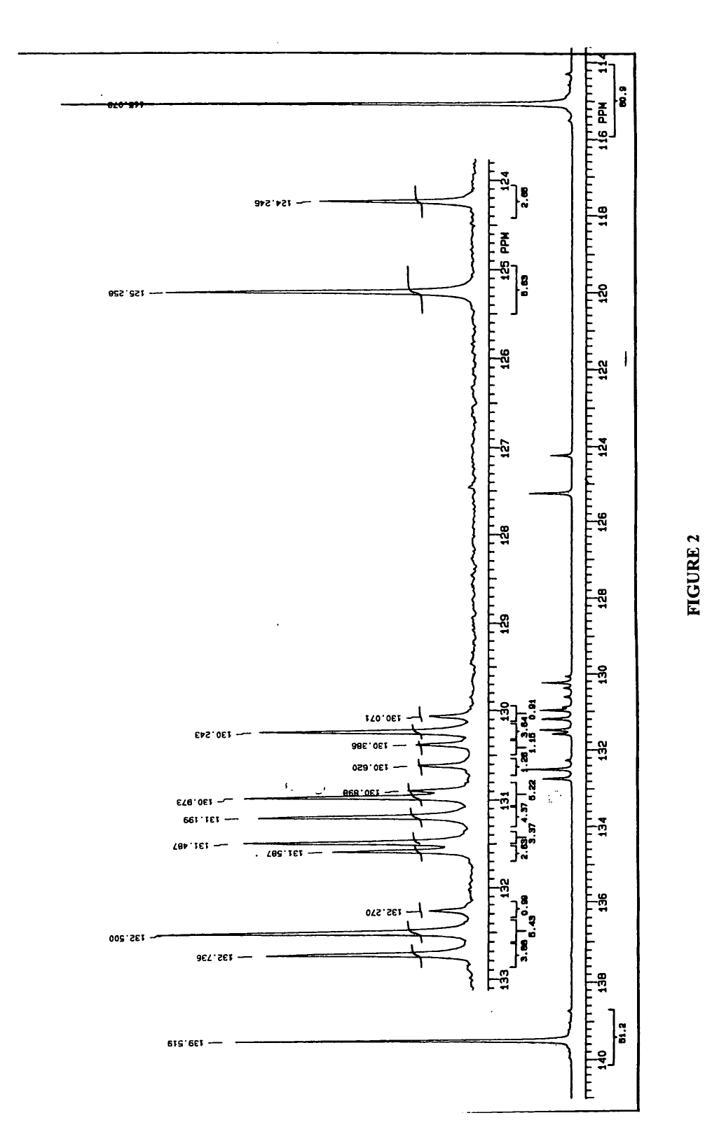

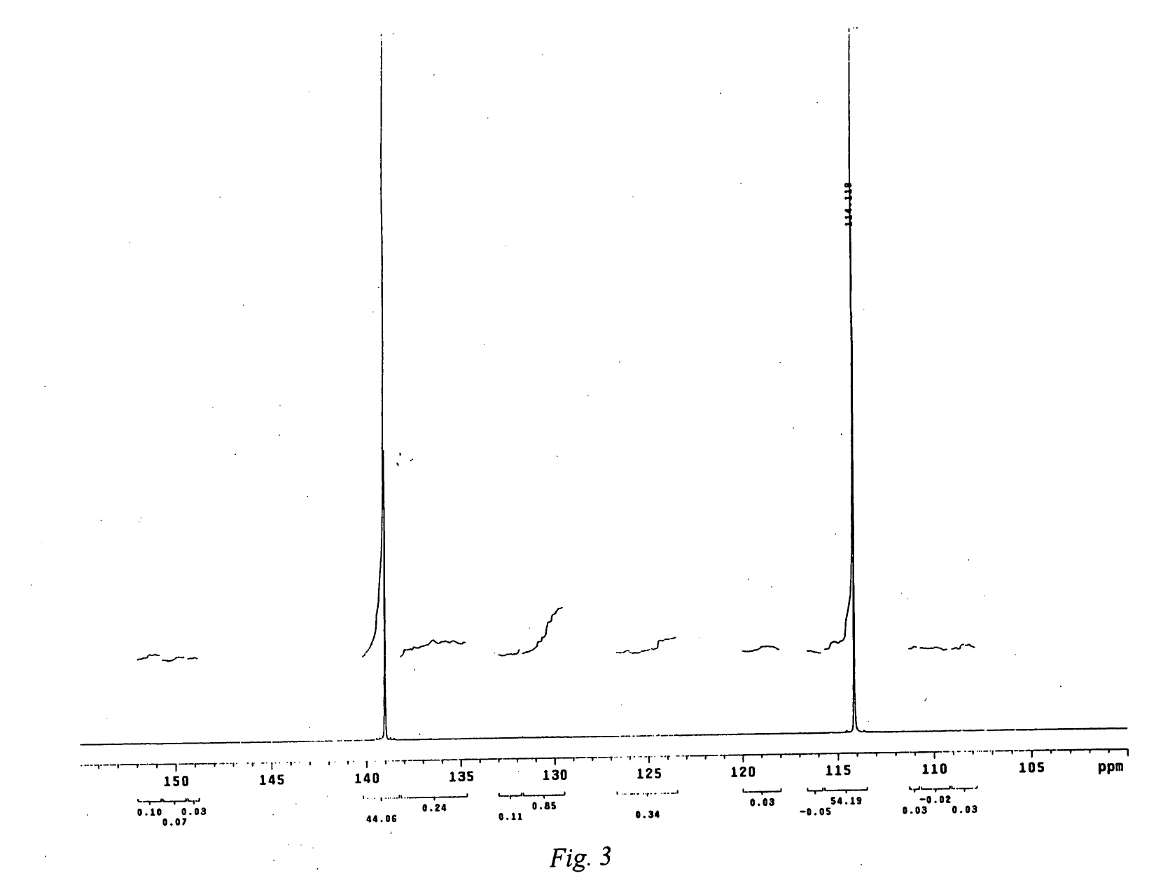

FIGS. 2 and 3 contain ¹³C NMR data that compares two processes that were used to produce a polyalphaolefin. FIG. 2 illustrates ¹³C NMR data for a monomer recycle stream from a prior art process that uses a boron trifluoride/alcohol catalyst. As will be understood by those of skill in the art, isomers of the feed monomer used to produce the polyalphaolefin can be seen in the range of about 130 to 133 ppm in FIG. 2, which is, indicative of migration of the carbon-carbon double bond. FIG. 3 illustrates ¹³C NMR data from the recycle stream used in a process embodiment of the present invention. As can be seen in FIG. 3, in a comparable range, essentially no isomers of the monomer were formed.

In the description above, like parts are marked throughout the specification and drawings with the same reference numerals, respectively. The drawing figures are not necessarily to scale. Certain features of the invention may be shown exaggerated in scale or in somewhat schematic form and some details of conventional elements may not be shown in the interest of clarity and conciseness. The present disclosure is susceptible to embodiments of different forms. There are shown in the drawings, and herein are described in detail, specific embodiments of the present disclosure with the understanding that the present disclosure is to be considered an exemplification of the principles of the invention, and is not intended to limit the invention to that illustrated and described herein. It is to be fully recognized that the different teachings of the embodiments discussed above can be employed separately or in any suitable combination to produce desired results. Specifically, the method and system of the present invention disclosed herein to add high shear mixing to an ionic liquid catalyzed reaction can be used with any suitable ionic liquid catalyzed reaction wherein the reaction product contains a converted chemical reactant. In a desirable embodiment, the method and system to add high shear mixing to an ionic liquid catalyzed reaction of the present disclosure is for an oligomerization reaction for producing PAO from monomer or mixtures thereof, in the presence of an ionic liquid based catalyst system and the detailed description above is focused on this embodiment but with the understanding that the present invention can have broader applications including such reactions as a Friedel-Crafts alkylation. Although only a few embodiments of the present invention have been described herein, it should be understood that the present disclosure can be embodied in many other specific forms without departing from the spirit or the scope of the present disclosure. Any examples included are to be considered as illustrative and not restrictive, and the disclosure is not to be limited to the details given herein, but can be modified within the scope of the appended claims along with their full scope of equivalents.

That which is claimed is:

1. A method to produce an oligomer comprising the steps of:
(a) contacting a monomer comprising an alpha olefin with an ionic liquid catalyst to produce a reactor effluent stream comprising at least a portion of the monomer, the oligomer, the ionic liquid catalyst, and combinations thereof;
(b) separating at least a portion of the monomer from the oligomer and the ionic liquid catalyst, and
(c) recycling the at least a portion of the monomer for use in the step of contacting the monomer with the ionic liquid catalyst, the at least a portion of the monomer being substantially non-isomerized through the step of contacting the monomer with the ionic liquid catalyst.

2. The method of claim 1, wherein the alpha olefin has from 4 to 20 carbon atoms.

3. The method of claim 1, wherein the alpha olefin is 1-decene, 1-dodecene, or combinations thereof.

4. The method of claim 1, wherein the monomer comprises more than one alpha olefin.

5. The method of claim 1, wherein the ionic liquid catalyst comprises a metal halide and an alkyl-containing amine hydrohalide salt.

6. The method of claim 1, wherein the step of contacting the monomer comprises contacting the monomer with the ionic liquid catalyst, and oxygen, water, or combinations thereof

7. The method of claim 1, further comprising the step of hydrogenating the oligomer to produce a polyalphaolefin product.

8. A method to produce an oligomer comprising the steps of:
(a) contacting a monomer comprising an alpha olefin with an ionic liquid catalyst to produce a reactor effluent stream comprising at least a portion of the monomer, a dimer, the oligomer, the ionic liquid catalyst, and combinations thereof,
(b) separating at least a portion of the monomer and the dimer from the oligomer and the ionic liquid catalyst; and
(c) recycling the at least a portion of the monomer and the dimer for use in the step of contacting the monomer with the ionic liquid catalyst, the at least a portion of the monomer being substantially non-isomerized through the step of contacting the monomer with the ionic liquid catalyst.

9. The method of claim 8, wherein the step of separating the at least a portion of the monomer and the dimer from the oligomer and the ionic liquid catalyst is performed using more than one separation step.

10. The method of claim 8, wherein the monomer comprises more than one alpha olefin.

11. The method of claim 8, wherein the alpha olefin is 1-decene, 1-dodecene, or combinations thereof:

12. The method of claim 8, wherein the ionic liquid catalyst comprises a metal halide and an alkyl-containing amine hydrohalide salt.

13. The method of claim 8, wherein the step of contacting the monomer comprises contacting the monomer with the ionic liquid catalyst, and oxygen, water, or combinations thereof

14. The method of claim 8, further comprising the step of hydrogenating the oligomer to produce a polyalphaolefin product.

15. A method to produce an oligomer comprising the steps of
(a) contacting a monomer comprising an alpha olefin with an ionic liquid catalyst to produce a reactor effluent stream comprising at least a portion of the monomer, the oligomer, the ionic liquid catalyst, and combinations thereof;
(b) separating the at least a portion of the monomer from the oligomer and the liquid ionic catalyst;
(c) separating at least a portion of the liquid ionic catalyst from the oligomer, and
(d) recycling the at least a portion of the liquid ionic catalyst and the at least a portion of the monomer for use in the step of contacting the monomer with the ionic liquid catalyst, the at least a portion of the monomer being substantially non-isomerized through the step of contacting the monomer with the ionic liquid catalyst.

16. The method of claim 15, wherein the alpha olefin comprises from 4 to 20 carbon atoms.

17. The method of claim 15, wherein the alpha olefin is 1-decene, 1-dodecene, or combinations thereof:

18. The method of claim 15, further comprising the step of hydrogenating the oligomer to produce a polyalphaolefin product.

19. The method of claim 15, wherein the ionic liquid catalyst comprises a metal halide and an alkyl-containing amine hydrohalide salt.

20. The method of claim 15, wherein the step of contacting the monomer comprises contacting the monomer with the ionic liquid catalyst, and oxygen, water, or combinations thereof
